(19) United States
(12) Patent Application Publication
(10) Pub. No.: US 2016/0022681 A1
(43) Pub. Date: Jan. 28, 2016

(54) METHODS OF INCREASING LYMPHATIC TRANSPORT

(71) Applicant: UNIVERSITY OF ROCHESTER, Rochester, NY (US)

(72) Inventors: Echoe Bouta, Rochester, NY (US); Ronald Wood, Rochester, NY (US); Edward M. Schwarz, Rochester, NY (US)

(73) Assignee: UNIVERSITY OF ROCHESTER, Rochester, NY (US)

(21) Appl. No.: 14/378,764

(22) PCT Filed: Feb. 11, 2013
(86) PCT No.: PCT/US13/25516

§ 371 Date: Aug. 14, 2014

Related U.S. Application Data
(60) Provisional application No. 61/598,615, filed on Feb. 14, 2012.

(51) Int. Cl. A61K 31/519; A61K 49/00

(52) U.S. Cl. CPC A61K 31/519; A61K 49/0034

(57) ABSTRACT

Provided herein are methods of treating a lymphatic transport dysfunction or condition in a subject. Also provided are methods of preventing or reducing a flare in a subject with a lymphatic transport dysfunction or condition. Further provided are methods of monitoring lymphatic transport in a subject and methods of selecting a treatment protocol for a subject with a lymphatic transport dysfunction or condition. Further provided are methods of detecting a flare in a subject with a lymphatic transport dysfunction or condition.

### RELATED APPLICATIONS

This application claims the benefit of U.S. Application No. 61/598,615, filed on Feb. 14, 2012, which is hereby incorporated herein in its entirety.

### STATEMENT REGARDING FEDERALLY FUNDED RESEARCH

This invention was made with government funding under Grant Nos. T32 AR53459, AI78907, AR61307, RO1 AR56702, UL1 RR024160, and RO1 DK075036 from the National Institutes of Health. The government has certain rights in this invention.

### BACKGROUND

Lymphatic vessels are present in almost all tissues of the body. They are composed of an extensive network of thin-walled vessels that drain protein-rich lymph from extracellular spaces. Under normal conditions, the major functions of the lymphatic system include maintenance of tissue fluid homeostasis, absorption of fatty acids, and mediation of the afferent immune response. Recent studies have provided increasing evidence that the lymphatic system also plays key roles in disease processes such as cancer metastasis, lymphedema, obesity, and inflammation.

### SUMMARY

Provided herein are methods of treating a lymphatic transport dysfunction or condition in a subject. The methods comprise selecting a subject with a lymphatic transport dysfunction or condition and administering to the subject a phosphodiesterase (PDE) inhibitor. Also provided are methods of preventing or reducing a flare in a subject with a lymphatic transport dysfunction or condition. The methods comprise selecting a subject at risk of a flare and administering to the subject a PDE inhibitor.

Also provided are methods of monitoring lymphatic transport in a subject. The methods comprise administering an imaging agent and a PDE inhibitor to a subject with a lymphatic transport dysfunction or condition and detecting lymphatic transport of the imaging agent.

Further provided are methods of selecting a treatment protocol for a subject with a lymphatic transport dysfunction or condition. The methods comprise administering to the subject a PDE inhibitor and an imaging agent, monitoring a level of lymphatic transport of the imaging agent in the subject, and selecting the treatment protocol based on the level of lymphatic transport in the subject.

Further provided are methods of detecting a flare in a subject with a lymphatic transport dysfunction or condition. The methods comprise selecting a subject with a lymphatic transport dysfunction or condition, administering to the subject a PDE inhibitor and an imaging agent, and monitoring a level of lymphatic transport of the imaging agent in the subject. A reduced level of lymphatic transport of the imaging agent as compared to a control level indicates a flare. An increased level of lymphatic transport of the imaging agent as compared to a control level indicates an early stage of inflammation.

The details of one or more embodiments are set forth in the accompanying drawings and the description below. Other features, objects, and advantages will be apparent from the description and drawings, and from the claims.

### DESCRIPTION OF DRAWINGS

FIG. 1 shows sildenafil increases indocyanine green (ICG) uptake into lymphatic vessels. Saline (A, left panel) or sildenafil (12 mg/kg) (B, right panel) was injected intraperitoneally (I.P.) (0.5 mL) 20 minutes after injection of ICG. Images were taken 20 minutes after treatment.

### DETAILED DESCRIPTION

Lymphatic transport dysfunctions or conditions include, but are not limited to, lymphedema, rheumatoid arthritis, Crohn's disease, multiple sclerosis, and psoriasis. There is currently no explanation for the severity of the dysfunctions or conditions and the sudden onset of the dysfunctions or conditions, commonly referred to as a flare.

Provided herein are methods of treating a lymphatic transport dysfunction or condition in a subject. The methods comprise selecting a subject with a lymphatic transport dysfunction or condition and administering to the subject a phosphodiesterase (PDE) inhibitor. Without intending to be limited by theory, administration of a phosphodiesterase inhibitor to a subject with a lymphatic transport dysfunction or disorder can, for example, dilate parallel lymphatic vessels allowing for an increase in lymphatic flow, thus treating a lymphatic transport dysfunction or condition.

Also provided is a PDE inhibitor for use in treating a lymphatic transport dysfunction or condition in a subject, wherein the treatment comprises (a) selecting a subject with a lymphatic transport dysfunction or condition and (b) administering to the subject the phosphodiesterase (PDE) inhibitor.

As utilized throughout, lymphatic transport can include, but is not limited to, lymphatic clearance, uptake of molecules, fluids or dyes (for example, a cyanine dye, such as, indocyanine green (ICG)), and lymphatic contraction. Therefore, a lymphatic transport dysfunction or condition can be characterized by, for example, and not to be limiting, hindered lymphatic clearance, inhibited lymphatic uptake, and/or decreased lymphatic contraction.

Selecting a subject with a lymphatic transport dysfunction or condition can, for example, comprise identifying a subject with symptoms of the lymphatic transport dysfunction or condition (e.g., inflammation or swelling of surrounding tissues, fluid buildup, swollen lymph nodes, neuropathy, itchiness, and joint pain). Optionally, selecting a subject with a lymphatic transport dysfunction or disorder can comprise initially diagnosing a lymphatic transport dysfunction or condition by observation of one or more symptoms of a lymphatic transport dysfunction or condition.

Also provided are methods of preventing or reducing a flare in a subject with a lymphatic transport dysfunction or condition. The methods comprise selecting a subject at risk of a flare and administering to the subject a PDE inhibitor. Selecting a subject at risk of a flare can, for example, comprise identifying a subject at risk of developing a lymphatic transport dysfunction or condition or identifying a subject beginning to experience symptoms of the lymphatic transport dysfunction or condition (e.g., beginning to experience inflammation, accumulation of fluid, swelling of the lymph, swelling of the tissues, itchiness, and joint pain).

Further provided is a PDE inhibitor for use in preventing or reducing a flare in a subject with a lymphatic transport dysfunction or condition, wherein preventing or reducing a flare in the subject comprises (a) selecting a subject at risk of a flare and (b) administering to the subject the phosphodiesterase (PDE) inhibitor. Optionally, the lymphatic transport dysfunction or condition is selected from lymphedema or an immune mediated inflammatory disorder or condition. Lymphedema can, for example, be a primary or secondary lymphedema. A primary lymphedema can, for example, be an inherited disorder. A secondary lymphedema can, for example, be the result of an injury, a parasitic infection (e.g., filariasis), or a surgical procedure. Secondary lymphedemas are frequently seen after lymph node dissection, surgery, and/or radiation therapy, in which damage to the lymphatic system is caused during the treatment of cancer. Lymphedema is known in the art, see, e.g., Korpan et al., Am. J. Phys. Med. Rehabil. 90(5 Suppl. 1):S69-75 (2011); Murdaca et al., Am. J. Med. 125(2):134-40 (2012); Cavanaugh, J. Oncol. Pract. 7(2):89-93 (2011); Mehrara et al., Lymphat. Res. Biol. 9(3):159-67 (2011), Ferguson et al., Am. J. Med. Genet. 155A(11):2762-5 (2011), and Oremus et al., BMC Cancer 12(1):6 (2012). An immune mediated inflammatory disorder or condition can, for example, be selected from the group consisting of Crohn's disease, colitis, irritable bowel syndrome, pelvic pain syndromes (e.g., interstitial cystitis, endometroiosis, and fibromyalgia), psoriasis, multiple sclerosis, and arthritis. Immune mediated inflammatory disorders or conditions are known in the art, see, e.g., Cantaert et al., Arthritis Res. Ther. 12(5):219 (2010), Perera et al., Annu. Rev. Pathol. (2011), Kuck et al., Postgrad Med. J., 83:251-60 (2007), and Firestein and Corr, J. Rheumatol. 73:8-13 (2005). Multiple sclerosis is known in the art. See, e.g., Sormani et al., J. Neurol. Neurosurg. Psychiatry 70:494-9 (2001). Arthritis can, for example, be selected from the group consisting of rheumatoid arthritis (RA), osteoarthritis, and spondylitis (e.g., ankylosing spondylitis). The lymphatic transport disease or condition is not an erectile dysfunction.

The PDE inhibitor can be a selective or a non-selective PDE inhibitor. For example, the PDE inhibitor can be a PDE-1, a PDE-2, a PDE-3, or a PDE-4 inhibitor. Optionally, in any of the methods or uses provided herein, a PDE inhibitor can be administered to a subject, wherein the PDE inhibitor is not a PDE-4 inhibitor. Optionally, in any of the methods or uses provided herein, a PDE-4 inhibitor can be administered to a subject wherein the PDE inhibitor is not Apremilast. Optionally, in any of the methods or uses provided herein, a PDE inhibitor can be administered to the subject, wherein the PDE inhibitor is not a PDE-4 inhibitor, and wherein the lymphatic transport disorder is not rheumatoid arthritis. Optionally, in any of the methods or uses provided herein, a PDE inhibitor can be administered to the subject, wherein the PDE inhibitor is not a Apremilast, and wherein the lymphatic transport disorder is not rheumatoid arthritis.

Optionally, the PDE inhibitor is a PDE-5 inhibitor. The PDE-5 inhibitor can, for example, be selected from the group consisting of sildenafil, tadalafil, avanafil, lodenafil, mirodenafil, vardenafil, udenafil, icarrin, nitrosoprodenafil, acetifenafil, aildenafil (methisosildenafil), and sulfoaildenafil (thioaildenafil). The PDE-5 inhibitor can, for example, be a long acting PDE-5 inhibitor (e.g., tadalafil). The PDE-5 inhibitor can, for example, be a short acting PDE-5 inhibitor (e.g., sildenafil). PDE inhibitors are known, see, e.g., European Patent No. 0977756; U.S. Pat. No. 5,859,006; U.S. Pat. No. 5,874,437; U.S. Pat. No. 5,981,527; U.S. Pat. No. 6,037,346; U.S. Pat. No. 6,140,329; U.S. Pat. No. 6,143,746; U.S. Pat. No. 6,172,060; U.S. Pat. No. 6,362,178; U.S. Pat. No. 7,696,206; United States Publication No. 2003/0083228, which are herein incorporated by reference in their entireties.

Selection of the long acting or short acting PDE-5 inhibitor is made by one of the skill in the art based on the need. For example, for imaging uses, a short acting form is preferable, whereas, a long acting form is preferable for treatment of chronic disorder or prevention of a flare. Optionally, a combination of long and short acting can be used (e.g., at the first sign of a flare).

Also provided are methods of monitoring lymphatic transport in a subject with a lymphatic transport dysfunction or condition. The methods comprise administering an imaging agent and a PDE inhibitor to the subject and detecting lymphatic transport of the imaging agent. Detection of lymphatic transport can, for example, comprise visualizing the movement of the imaging agent through the lymphatic vessels.

Further provided is a PDE inhibitor for use in monitoring lymphatic transport in a subject, wherein the monitoring comprises: (a) administering an imaging agent and the phosphodiesterase (PDE) inhibitor to a subject with a lymphatic transport dysfunction or condition and (b) detecting lymphatic transport of the imaging agent.

Further provided are methods of selecting a treatment protocol (e.g., the type of PDE inhibitor or an amount of the PDE inhibitor) for a subject with a lymphatic transport dysfunction or condition. The methods comprise administering to the subject a PDE inhibitor and an imaging agent, monitoring the level of lymphatic transport of the imaging agent in the subject, and selecting a treatment protocol based on the level of lymphatic transport in the subject. A reduction or absence of lymphatic transport of the imaging agent as compared to a control indicates the need to select a different treatment protocol to increase the level of lymphatic transport (e.g., an increased amount of PDE inhibitor can be administered to the subject or a different PDE inhibitor can be administered to the subject to increase the level of lymphatic transport). An increase in the level of lymphatic transport of the imaging agent as compared to a control indicates that the current treatment protocol is sufficient. As used herein, a control can, for example, comprise a level of lymphatic transport observed in a normal, non-diseased subject that is administered the same treatment protocol.

Also provided is a PDE inhibitor for use in selecting a treatment protocol for a subject with a lymphatic transport dysfunction or condition, wherein the selection comprises (a) administering to the subject the PDE inhibitor and an imaging agent, (b) monitoring a level of lymphatic transport of the imaging agent in the subject, and (c) selecting the treatment protocol based on the level of lymphatic transport in the subject

Further provided are methods of detecting a flare in a subject with a lymphatic transport dysfunction or condition. The methods comprise selecting a subject with a lymphatic transport dysfunction or condition, administering to the subject a PDE inhibitor and an imaging agent, and monitoring a level of lymphatic transport of the imaging agent in the subject. A reduced level of lymphatic transport of the imaging agent as compared to a control indicates a lymphatic flare. An increased level of lymphatic transport of the imaging agent as compared to a control indicates an early stage of inflammation. As used herein, a control can comprise a level of lymphatic transport of an imaging agent in a normal, non-diseased subject or joint (or other uninvolved area or limb) that is administered a PDE inhibitor and an imaging agent. Optionally, a control can comprise a diseased subject that has previously been diagnosed with a late or early stage lymphatic flare, but is currently not experiencing a flare.

Also provided is a PDE inhibitor for use in detecting a flare in a subject with a lymphatic transport dysfunction or condition, wherein the detection comprises (a) selecting a subject with a lymphatic transport dysfunction or condition; (b) administering to the subject the phosphodiesterase (PDE) inhibitor and an imaging agent; and (c) monitoring a level of lymphatic transport of the imaging agent in the subject, wherein lymphatic transport of the imaging agent is reduced in a flare and is increased in an early stage of inflammation as compared to a control level

A lymphatic flare can, for example, be characterized by collapsed lymph node volume after a period of inflammation-related expansion, and an eventual failure of the lymphatic vessels to contract. An early stage of inflammation can, for example, be characterized by the beginning of transport dysfunction, i.e., an inability of the lymphatics to clear the fluid within and an increase in frequency of lymphatic pulsing.

Imaging agents are agents that can be visualized using clinical imaging techniques. Such agents can, for example, be selected from the group consisting of indocyanine green, methylene blue, a radioisotope, an echogenic gas-filled sphere, a liposome, a micelle, or a nanoparticle. Imaging agents can also include fluorescent dyes. Fluorescent dyes are known in the art. See, e.g., Te Velde et al., Eur. J. Surg. Oncol. 36(1):6-15 (2010); Levitus and Ranjit, Q. Rev. Biophys. 44(1):123-51 (2011); and Parish, Immunol. Cell Biol. 77(6):499-508 (1999). Lymphatic fluorescence is known in the art, see, e.g., United States Patent Publication Nos. 20080161744 and 20080125650; U.S. Pat. Nos. 5,948,763, 6,462,171, 6,713,450, 7,479,482, and 7,700,107; Maus et al., Head Neck 34(3):448-53 (2012); Kwon et al., Neurogastroenterol. Motil. (Published online Feb. 6, 2012); Lu et al., J. Biomed. Opt. 16(12):126002 (2011); and Azhdarinia et al., Mol. Imaging Biol. (Published online Dec. 13, 2011).

Methods for detecting the imaging agent can, for example, be selected from the group consisting of near-infrared (NIR) imaging, ultrasound imaging, and radiological imagining. Imaging methods are known in the art. See, e.g., Altinoglu and Adair, Wiley Interdiscip. Rev. Nanomed. Nanobiotechnol. 2(5):461-77. (2010); Dempsey et al., Ann. N.Y. Acad. Sci. 820:149-69 (1997); Linder, Prog. Cardiovasc. Dis. 44(2):111-20 (2001); Quinn et al., Can. J. Cardiol. 16(7):911-7 (2000); and Grenier et al., Semin. Nucl. Med. 41(1):45-60 (2011).

Provided herein are compositions containing the PDE inhibitors and a pharmaceutically acceptable carrier described herein. The herein provided compositions are suitable for administration in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the PDE inhibitors, to humans or other subjects. The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intradermally, intracavity (e.g., rectal, intravesical, lumen of vesical organs), transdermally, intrahepatically, intracranially, nebulization/inhalation, or by installation via bronchoscopy. Intradermal administration includes administration at a site that is afferent to the site of lymphatic transport dysfunction. Optionally, the composition is administered by oral inhalation, nasal inhalation, intranasal mucosal administration, or suppository. The composition can also be injected or infused, for example, at a site of inflammation, such as, for example, an inflamed joint. Administration of the compositions by inhalant can be through the nose or mouth via delivery by spraying or droplet mechanism, for example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g. a lymphatic transport dysfunction or condition). The term patient or subject includes human and veterinary subjects.

A subject at risk of developing a lymphatic transport dysfunction or condition can be genetically predisposed to the lymphatic transport dysfunction or condition, e.g., have a family history or have a mutation in a gene that causes or is linked to the lymphatic transport dysfunction or condition (e.g., mutations in FOXC2 or SOX18 are linked to lymphedema-distichiasis and hypotrichosis-lymphedema-telangiectasia, respectively), or show early signs or symptoms of the lymphatic transport dysfunction or condition. A person skilled in the art would be capable of identifying mutations in genes that cause or are linked to a lymphatic transport dysfunction or condition. A subject currently with a lymphatic transport dysfunction or condition has one or more than one symptom of the lymphatic transport dysfunction or condition (e.g., inflammation of the lymph, fluid buildup, swollen lymph nodes, swollen tissue, itchiness, and joint pain) and may have been diagnosed with the lymphatic transport dysfunction or condition.

The methods and agents as described herein are useful for both prophylactic and therapeutic treatment of the lymphatic transport dysfunction or condition or a flare thereof. For prophylactic use, a therapeutically effective amount of the agent (e.g., PDE inhibitor) described herein is administered to a subject prior to onset (e.g., before obvious signs of the lymphatic transport dysfunction or condition) or during early onset (e.g., upon initial signs and symptoms of the lymphatic transport dysfunction or condition). Prophylactic administration can occur for several days to years prior to the manifestation of symptoms of the lymphatic transport dysfunction or condition. Prophylactic administration can be used, for example, in the preventative treatment of subjects diagnosed with a genetic predisposition (e.g., mutations in FOXC2 or SOX18 are linked to lymphedema-distichiasis and hypotrichosis-lymphcdema-telangiectasia, respectively) to the lymphatic transport dysfunction or condition. Therapeutic treatment involves administering to a subject a therapeutically effective amount of the agents described herein after diagnosis or development of the lymphatic transport dysfunction or condition.

According to the methods taught herein, the subject is administered an effective amount of the agent (e.g., PDE inhibitor). The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the lymphatic transport dysfunction or condition are affected (e.g., reduced or delayed flare). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days, and may be continued indefinitely. Dose range should not exceed the maximally tolerated clinical dose, which can vary across compounds depending on potency, formulation, and duration of action. Effective doses can also be extrapolated from dose-response curves derived from in vitro or animal models.

The effective amount of the agent or pharmaceutically acceptable salts thereof as described herein may be determined by one of ordinary skill in the art and includes exemplary dosage amounts for a mammal of from about 0.001 to about 50 mg/kg of body weight of active agent per day, which can be administered in a single dose or in the form of individual divided doses, such as from 1 to 4 times per day. For example, the dosage can be from about 0.001 to about 0.01 mg/kg, from about 0.001 to about 0.5 mg/kg, from about 0.001 to about 0.75 mg/kg, from about 0.001 to about 1 mg/kg, from about 0.001 to about 2.5 mg/kg, from about 0.001 to about 5 mg/kg, from about 0.001 to about 7.5 mg/kg, from about 0.001 to 10 mg/kg of body weight of active agent per day. Alternatively, the dosage amount can also be from about 0.01 to about 0.1 mg/kg, from about 0.01 to about 0.2 mg/kg, from about 0.01 to about 0.3 mg/kg, from about 0.01 to about 0.4 mg/kg, from about 0.01 to about 0.5 mg/kg, from about 0.01 to about 0.6 mg/kg, from about 0.01 to about 0.7 mg/kg, from about 0.01 to about 0.8 mg/kg, from about 0.01 to about 0.9 mg/kg from about 0.01 to about 1.0 mg/kg, from about 0.01 to about 1.5 mg/kg, from about 0.01 to about 2.0 mg/kg, from about 0.01 to about 2.5 mg/kg, from about 0.01 to about 3.0 mg/kg, from about 0.01 to about 3.5 mg/kg, from about 0.01 to about 4 mg/kg, from about 0.01 to about 4.5 mg/kg, or from about 0.01 to about 5 mg/kg.

Alternatively, the dosage amount can be from about 0.01 mg to about 500 mg which can be administered in a single dose or in the form of individual divided doses, such as from 1 to 4 times per day. The dosage amount can also be administered, for example, in a single dose or in individual divided doses 1 to 4 times over a 36 hour period, 1 to 4 times over a 48 hour period, or 1 to 4 times over a 72 hour period to achieve a dosage amount of from about 0.01 mg to about 5 mg, from about 0.01 mg to about 7.5 mg, from about 0.01 mg to about 10 mg, from about 0.01 mg to about 15 mg, from about 0.01 mg to about 20 mg, from about 0.01 mg to about 25 mg, from about 0.01 mg to about 30 mg, from about 0.01 mg to about 40 mg, from about 0.01 mg to about 50 mg, from about 0.01 mg to about 60 mg, from about 0.01 mg to about 70 mg, from about 0.01 mg to about 80 mg, from about 0.01 mg to about 90 mg, from about 0.01 mg to about 100 mg, from about 0.01 mg to about 125 mg, from about 0.01 mg to about 150 mg, from about 0.01 mg to about 175 mg, from about 0.01 mg to about 200 mg, from about 0.01 mg to about 225 mg, from about 0.01 mg to about 250 mg, from about 0.01 mg to about 275 mg, from about 0.01 mg to about 300 mg, from about 0.01 mg to about 325 mg, from about 0.01 mg to about 350 mg, from about 0.01 mg to about 375 mg, 0.01 mg to about 400 mg, from about 0.01 mg to about 425 mg, from about 0.01 mg to about 450 mg, from about 0.01 mg to about 475 mg, or from about 0.01 mg to about 500 mg.

Alternatively, the dosage amount can be from about 0.1 mg to about 500 mg which can be administered in a single dose or in the form of individual divided doses, such as from 1 to 4 times per day. The dosage amount can also be administered, for example, in a single dose or in individual divided doses 1 to 4 times over a 36 hour period, 1 to 4 times over a 48 hour period, or 1 to 4 times over a 72 hour period to achieve a dosage amount of from about 0.1 mg to about 5 mg, from about 0.1 mg to about 7.5 mg, from about 0.1 mg to about 10 mg, from about 0.1 mg to about 15 mg, from about 0.1 mg to about 20 mg, from about 0.1 mg to about 25 mg, from about 0.1 mg to about 30 mg, from about 0.1 mg to about 40 mg, from about 0.1 mg to about 50 mg, from about 0.1 mg to about 60 mg, from about 0.1 mg to about 70 mg, from about 0.1 mg to about 80 mg, from about 0.1 mg to about 90 mg, from about 0.1 mg to about 100 mg, from about 0.1 mg to about 125 mg, from about 0.1 mg to about 150 mg, from about 0.1 mg to about 175 mg, from about 0.1 mg to about 200 mg, from about 0.1 mg to about 225 mg, from about 0.1 mg to about 250 mg, from about 0.1 mg to about 275 mg, from about 0.1 mg to about 300 mg, from about 0.1 mg to about 325 mg, from about 0.1 mg to about 350 mg, from about 0.1 mg to about 375 mg, 0.1 mg to about 400 mg, from about 0.1 mg to about 425 mg, from about 0.1 mg to about 450 mg, from about 0.1 mg to about 475 mg, or from about 0.1 mg to about 500 mg.

In another example, the agent can be administered orally at a dosage amount selected from the group consisting of from about 0.1 mg to about 5 mg, from about 0.1 mg to about 10 mg, from about 0.1 mg to about 25 mg and from about 0.1 mg to about 50 mg.

Alternatively, the agent can be injected at a local site of inflammation, for example, an inflamed joint, at a dosage amount of 0.1 mg to about 1 mg or from about 0.1 mg to 5 mg.

Those of skill in the art will understand that the specific dose level and frequency of dosage for any particular subject may be varied and will depend upon a variety of factors, including the activity of the specific agent employed, the metabolic stability and length of action of that agent, the species, age, body weight, general health, sex and diet of the subject, the mode and time of administration, rate of excretion, and severity of the particular condition. Additional guidance can be found in the literature for appropriate dosages for given classes of pharmaceutical products.

As used herein the terms treatment, treat, or treating refers to a method of reducing an effect of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Sildenafil Increased Indocyanine Green Uptake in the Lymphatic System

To elucidate the cellular and molecular mechanisms responsible for arthritic flare, a three dimensional (3D) contrast enhanced-magnetic resonance imaging (CE-MRI) for the mouse was developed, and this approach was used to evaluate the natural history of inflammatory-erosive arthritis in various murine models (Guo et al., Arthritis Rheum. 60(9):2666-76 (2009); Proulx et al., Ann. N.Y. Acad. Sci. 1117:106-23 (2007); Proulx et al., Arthritis Rheum. 58(7):2019-29 (2008); Proulx et al., Arthritis Rheum. 56(12):4024-37 (2007); Zhang et al., Arthritis Res. Ther. 9(6):R118 (2007)). The results of these studies produced several findings that the changes in the volume and contrast enhancement (CE) of draining lymph nodes preceded arthritic flare in adjacent joints (see, e.g., U.S. Ser. No. 12/299,171). Another seminal discovery was the observation that knee arthritis in the tumor necrosis factor transgenic (TNF-Tg) mouse model of RA is asymmetric in which the unaffected knee drains to an expanded-contrast enhancing popliteal lymph node (PLN), while the contralateral knee with severe inflammatory-erosive arthritis is adjacent to a much smaller PLN that fails to take up Gd-DTPA (Li et al., J. Immunol. 184(11):6142-50 (2010)). To further investigate these findings, a prospective study was performed in which hTNF-Tg mice with bilateral ankle arthritis were followed with CE-MRI every 2-weeks until they presented with knee synovitis, which revealed two distinct phases of disease progression. The first, characterized as the PLN “expansion” phase, is associated with increased, but relatively stable synovial volumes without bone erosions, and large lymphatic draining capacity (LNcap) values, which indicate an expanded, fluid-filled node. Subsequently, a yet to be identified event triggers the PLN “collapse” phase, in which LNcap values decrease rapidly due to parallel reductions in both PLN volume and CE, while synovitis worsens, as highlighted by higher synovial volume (SynVol) values. Consistent with synovitis presentation, knees that drain to an expanding PLN have no evidence of focal erosions, whereas knees adjacent to collapsed PLN display extensive bone loss (Zhou et al., Arthritis & Rheumatism 62(7):1881-9 (2010)).

Simultaneously, the role of lymphangiogenesis and lymphatic flow during arthritis initiation and progression in murine models of RA was investigated (Guo et al., Arthritis Rheum. 60(9):2666-76 (2009); Proulx et al., Ann. N.Y. Acad. Sci. 1117:106-23 (2007); Zhang et al., Arthritis Res. Ther. 9(6):R118 (2007); Li et al., J. Immunol. 184(11):6142-50 (2010); Zhou et al., Arthritis & Rheumatism 62(7):1881-9 (2010); Telinius et al., Heart Circul. Physiol. 299:H811-8 (2010)). This research produced several findings, most notably that the rate and direction of pulsation of lymphatic vessels as well as apparent lymph flow in the lymphatic vessels changes dramatically during the acute and chronic phases of inflammatory-erosive arthritis. The alterations in lymphatic clearance precede flare and using agents that promote lymphatic transport is beneficial in the treatment of rheumatoid arthritis (RA) patients. Several drugs have been shown to increase lymphatic pulsing and/or flow in ex vivo studies including: norepinephrine, doxium, hyaluronidase, substance P, endothelin-1, and U46619 (Telinius et al., Heart Circul. Physiol. 299:H811-8 (2010); Amerini et al., Lymphatic Res. Biol. 2(1):2-10 (2004); Repa et al., J. Cardiovasc. Pharmacol. 16(2):286-91 (1990)).

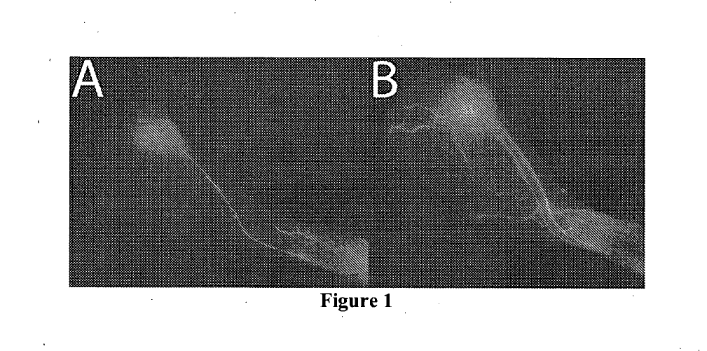

To determine if phosphodiesterase (PDE) inhibitors were capable of increasing lymphatic transport, near-infrared (NIR) imaging of indocyanine green ICG) transport was used. ICG (20 μl of 0.1 mg ICG/ml water) was injected into the footpad Of the animal Where it is taken up by the lymphatic system. The extremity was illuminated with near infrared light (generated with a tungsten halogen source) resulting in near infrared fluorescence at a longer wavelength that was recorded with a near-infrared sensitive camera (Prosilica GC1380; Allied Vision Technologies; Stadtroda, Germany). Excitation and emission wavelengths were separated using Semrock filters (IDEX; Lake Forest, Ill.) from the ICG-A filter set. To determine if PDE inhibitors Were capable of increasing lymphatic transport, sildenafil (12 mg/kg), a PDE5 antagonist, was injected intraperitoneally with ICG into the footpad of a mouse. Saline was used a control. After 20 minutes, images of the lymphatic vessels were taken which demonstrated that sildenafil increased ICG uptake into the lymphatic system (FIG. 1, right panel).

### Example 2

### The Effects of PDE5 Antagonists on Murine Models of Irritable Bowel Syndrome and Crohn's Disease

A method for cannulation of the mouse thoracic lymph duct (M. Ionac et al., J. Immunol. Meth. 202:35-40 (1997)) has been adapted to study acute pharmacologic effects on lymphatic transport from the intestine. A mouse is offered several milliliters (mis) of cream or sweetened condensed milk an hour before surgery, or is administered 1 ml by gavage (7.5% milk fat), anesthetized with isoflurane or urethane, and restrained on its right side on a surface warmed to 37-39° C. Murine models for use in studying the effects of PDE5 antagonists can, for example, include murine models disclosed in Table 1 of Pizarro et al., Trends Mol. Med. 9(5):218-22 (2003), which is incorporated herein by reference in its entirety.

An incision is made along the abdomen; the spleen, liver and stomach are retracted cranially and the left kidney and adrenal glands are retracted towards the tail. The aorta is exposed by cutting through the dorsal parietal peritoneum, exposing the adjacent cisterna chyli and thoracic lymphatic duct near the lumbar or superior mesenteric artery.

This procedure is done under an operating room microscope equipped with a digital camera providing a continuous stream of images at 10 frames per second or greater. Administration of a lipid material conjugated with a near infrared fluorescent dye facilitates visualization at depth and reduces the amount of dissection required for clear visualization and identification of lymphatic structures arising from visceral organs of the pelvis. Indocyanine green may be injected into the bowel wall; alternatively, fluorescent (preferably near infrared) antibodies to relevant cytokines (e.g., tumor necrosis factor alpha, interleukin-1, and interferon-gamma) may be administered to the bowel wall, and transport observed from the bowel to the thoracic duct. Systemic or topical administration of PDE5 antagonists produces dilation of the lymphatic vessels and change in lymphatic transport from the bowel.

These procedures are performed in control animals and in animals displaying altered bowel function as a result of enhanced production of cytokines such as tumor necrosis factor alpha. Alternatively, animals may be challenged with chemicals (e.g., 3% dextran sodium sulfate in drinking water; dinitrobenzene sulfuric acid) to produce acute bowel injury.

1. A method of treating a lymphatic transport dysfunction or condition in a subject, the method comprising (a) selecting a subject with a lymphatic transport dysfunction or condition and (b) administering to the subject a phosphodiesterase (PDE) inhibitor.

2. The method of claim 1, wherein the lymphatic transport dysfunction or condition is selected from lymphedema or an immune mediated inflammatory disorder or condition.

3-9. (canceled)

10. The method of claim 1, wherein the PDE inhibitor is a PDE-5 inhibitor.

11. The method of claim 10, wherein the PDE-5 inhibitor is selected from the group consisting of sildenafil, tadalafil, avanafil, lodenafil, mirodenafil, vardenafil, udenafil, icariin, nitrosoprodenafil, acetildenafil, aildenafil, and sulfoaildenafil.

12-13. (canceled)

14. A method of preventing or reducing a flare in a subject with a lymphatic transport dysfunction or condition, the method comprising (a) selecting a subject at risk of a flare and (b) administering to the subject a phosphodiesterase (PDE) inhibitor.

15. The method of claim 14, wherein the PDE inhibitor is a PDE-5 inhibitor.

16. The method of claim 15, wherein the PDE-5 inhibitor is selected from the group consisting of sildenafil, tadalafil, avanafil, lodenafil, mirodenafil, vardenafil, udenafil, icariin, nitrosoprodenafil, acetildenafil, aildenafil, and sulfoaildenafil.

17.-18. (canceled)

19. A method of monitoring lymphatic transport in a subject, the method comprising (a) administering an imaging agent and a phosphodiesterase (PDE) inhibitor to a subject with a lymphatic transport dysfunction or condition and (b) detecting lymphatic transport of the imaging agent.

20. (canceled)

21. (canceled)

22. The method of claim 19, wherein the lymphatic transport dysfunction or condition is selected from lymphedema or an immune mediated inflammatory disorder or condition.

23. (canceled)

24. (canceled)

25. (canceled)

26. (canceled)

27. (canceled)

28. (canceled)

29. (canceled)

30. The method of claim 19, wherein the PDE inhibitor is a PDE-5 inhibitor.

31. The method of claim 30, wherein the PDE-5 inhibitor is selected from the group consisting of sildenafil, tadalafil, avanafil, lodenafil, mirodenafil, vardenafil, udenafil, icariin, nitrosoprodenafil, acetildenafil, aildenafil, and sulfoaildenafil.

32. (canceled)

33. (canceled)

34. A method of selecting a treatment protocol for a subject with a lymphatic transport dysfunction or condition, the method comprising (a) administering to the subject a PDE inhibitor and an imaging agent, (b) monitoring a level of lymphatic transport of the imaging agent in the subject, and (c) selecting the treatment protocol based on the level of lymphatic transport in the subject.

35. The method of claim 34, wherein the lymphatic transport dysfunction or condition is selected from lymphedema or an immune mediated inflammatory disorder or condition.

36. (canceled)

37. (canceled)

38. (canceled)

39. (canceled)

40. (canceled)

41. (canceled)

42. (canceled)

43. The method of claim 34, wherein the PDE inhibitor is a PDE-5 inhibitor.

44. The method of claim 43, wherein the PDE-5 inhibitor is selected from the group consisting of sildenafil, tadalafil, avanafil, lodenafil, mirodenafil, vardenafil, udenafil, icariin, nitrosoprodenafil, acetildenafil, aildenafil, and sulfoaildenafil.

45. (canceled)

46. (canceled)

47. (canceled)

48. (canceled)

49. A method of detecting a flare in a subject with a lymphatic transport dysfunction or condition, the method comprising (a) selecting a subject with a lymphatic transport dysfunction or condition; (b) administering to the subject a phosphodiesterase (PDE) inhibitor and an imaging agent; and (c) monitoring a level of lymphatic transport of the imaging agent in the subject, wherein lymphatic transport of the imaging agent is reduced in a flare and is increased in an early stage of inflammation as compared to a control level.

50. The method of claim 49, wherein the lymphatic transport dysfunction or condition is selected from lymphedema or an immune mediated inflammatory disorder or condition.

51. (canceled)

52. (canceled)

53. (canceled)

54. (canceled)

55. (canceled)

56. (canceled)

57. (canceled)

58. The method of claim 49, wherein the PDE inhibitor is a PDE-5 inhibitor.

59. The method of claim 58, wherein the PDE-5 inhibitor is selected from the group consisting of sildenafil, tadalafil, avanafil, lodenafil, mirodenafil, vardenafil, udenafil, icariin, nitrosoprodenafil, acetildenafil, aildenafil, and sulfoaildenafil.

60. (canceled)

61. (canceled)

62. (canceled)

63. (canceled)

64. (canceled)

65. (canceled)

66. The method of claim 1, wherein the PDE inhibitor is administered at a dosage amount selected from the group consisting of from about 0.1 mg to about 5 mg, from about 0.1 mg to about 10 mg, from about 0.1 mg to about 25 mg and from about 0.1 mg to about 50 mg.

67. The method of claim 66, wherein the PDE inhibitor is administered orally.

68. The method of claim 66, wherein the subject has joint inflammation or joint paint, wherein the dosage amount is from about 0.1 mg to about 1 mg or from about 0.1 mg to about 5 mg, and wherein the inhibitor is injected into the joint of the subject.

69. The method of claim 66, wherein the PDE inhibitor is administered in a single dose.

70. The method of any of claim 66 wherein the PDE inhibitor is administered in individual divided doses.

71-80. (canceled)
